(19) United States
(12) Patent Application Publication
(10) Pub. No.: US 2005/0256447 A1
(43) Pub. Date: Nov. 17, 2005

(54) Control of liquid flow into or out of a human or animal body

(76) Inventors: Margaret Pamela Richardson, Saundersfoot (GB); Philip Richardson, Saundersfoot (GB)

Correspondence Address: BAKER & DANIELS LLP, 300 NORTH MERIDIAN STREET, SUITE 2700, INDIANAPOLIS, IN, 46204, US

(21) Appl. No.: 10/517,848

(22) PCT Filed: Jun. 16, 2003
(86) PCT No.: PCT/GB03/02579

§ 371 Date: Feb. 14, 2005

(30) Foreign Application Priority Data
Jun. 14, 2002 (GB) 0213596.0
Nov. 13, 2002 (GB) 0226436.4

(51) Int. Cl. A61M 31/00

(52) U.S. Cl. 604/65; 604/544; 604/318

(57) ABSTRACT

Apparatus for controlling the operation of a catheter, the apparatus including an inlet for receiving the output end of a catheter and an outlet for discharging liquid received from the catheter, valve means between the inlet and the outlet for controlling the flow of liquid through the apparatus, sensing means for sensing one or more properties of the liquid in the apparatus, and control means for controlling operation of the valve means in response to a sensed property of the liquid, such as pressure, and/or in response to a predetermined criteria, such as a time interval having elapsed.

This invention relates to the control of fluid flow into or out of the human or animal body and in particular, but not exclusively, to the control of the flow of urine along a catheter for draining urine from a bladder. The invention also extends to the control of the flow of fluid into a patient's body, e.g. by I.V. infusion.

Catheter associated urinary tract infections account for a large number of hospital acquired infections. In one study, forty four percent of hospitalised patients with catheters were found to develop significant bacteruria within 72 hours of catheterisation. Some of these infections result in complications including pylonephritis, epididymitis, abscess formation and chronic renal failure. This leads to catheter blockage which represents one of the most common complications associated with such infections. Reference is directed to Stickler D J, Morris N S, Winters C (1999) Simple Physical Model to study formation and physiology of biofllms on urethral catheters. Methods in Enzymology 310:494-501 for a discussion of these issues.

Attempts have been made to improve the methods for the prevention, control and treatment of catheter associated infections such as by the use of a closed drainage system, strict observance of aseptic techniques in the handling of catheters, the use of cranberry juice drinks and the production of catheters containing antimicrobial substances.

Once an indwelling device, catheter or prosthesis becomes infected, the associated micro-organisms show remarkable resistance to both host defences and antimicrobial agents. Therefore, although all of these procedures have been shown to either reduce the incidence of—or delay the onset of—catheter associated infection, they have been unable to eradicate it.

Another commonly used method to combat infections and related blockage complications is irrigation of the urinary catheter with various solutions. However, the application of numerous agents using a variety of irrigation methods has resulted in a confusing picture, both in terms of their efficiency and clinical applications.

The building up of encrustation and urease activity results in an increase in urine alkalinity, and it has been shown in a study examining the pH encrustation that patients with a mean urinary pH below 6.8 had minute traces of encrustation, while patients above pH 6.8 had considerably more.

It has been found that periodic exercise of the bladder by allowing it to fill and empty normally is beneficial in an otherwise continuous drainage regime, in that normal bladder function is preserved and the average pH of the urine is lowered. Catheter encrustation is often found to be associated with a high urinary pH and the practice of testing the urine for pH level to identify patients most at risk of catheter encrustation has been shown to be beneficial in the management of such patients to avoid encrustation.

In addition to use of catheters on hospitalised patients, where the catheter may only be in place for a relatively short period, many catheters are used for much longer term applications outside the hospital, for example for users who are otherwise incontinent. In such applications, the user is permanently catheterised, with each catheter being withdrawn and replaced by a new catheter when the original has reached the end of its useful life. In beneficial conditions, where there are no exacerbating conditions such as low pH or greater than normal microbial activity, a conventional catheter might be expected to have a service life of three or four weeks. In extreme conditions where the urine is alkaline and/or there is greater than usual microbial activity the catheter may become blocked with matter within only a few hours.

The removal and reinsertion of a catheter can be distressing and uncomfortable for any user and so there is a need to prolong the effective life of a catheter as much as possible so as to reduce the frequency with which they need to be changed. Furthermore, withdrawing and reinsertion needs to be done by a medically qualified person with the attendant costs involved and so there is also a human resource and financial saving aspect, for all users.

The discomfort and distress experienced becomes substantially greater if there is significant encrustation of minerals such as calcium and magnesium ammonium phosphate crystals on the catheter because this is similar in texture to fine granules or sharp sand. Moreover, should a catheter become blocked, this will normally require hospitalisation of the user with attendant distress and expense.

Accordingly there is a major need for a catheter system in which the effective life of a catheter before it requires replacement can be prolonged and also for a system in which the occurrence of blockages is avoided or reduced, and/or a system in which the likely occurrence of a blockage or other adverse condition can be predicted and flagged, to allow timely preventive or remedial action to be taken.

WO 90/07353 discloses a controllable catheter tube for bladder cycling, in which the flow of urine through a catheter is controlled in response to a sensed pressure or an elapsed time. There is also reference to an embodiment in which a device infuses medication in response to an electrochemical transducer, but none of the illustrated embodiments suggest controlling the catheter in response to an electrochemical parameter instead of time or pressure.

Accordingly, in one aspect, this invention provides apparatus for controlling the operation of a catheter, the apparatus including an inlet for receiving the output end of a catheter and an outlet for discharging liquid received from the catheter, valve means between the inlet and the outlet for controlling the flow of liquid through the apparatus, sensing means for sensing one or more properties of the liquid in the apparatus, and control means for controlling operation of the valve means in response to a sensed property of the liquid, such as pressure, and/or in response to a predetermined criteria, such as a time interval having elapsed.

In another aspect, this invention provides apparatus for controlling the flow of fluid to or from a human or animal body, the apparatus including a flow passage having an inlet and an outlet, respective spaced flow control means disposed between the inlet and the outlet for controlling the flow of liquid through the apparatus and defining between them at least one chamber region, at least one sensing means for sensing one or more properties of the liquid in or adjacent the apparatus in use, and control means for controlling operation of the flow control means in response to at least one of:
(i) a sensed property of the liquid, and
(ii) elapse of a predetermined time interval.

The apparatus may have more than one such chamber disposed between spaced flow control means, or it may simply comprise first and second flow control means defining a single chamber.

Preferably, the inlet for receiving the output end of the catheter and the outlet for discharging it is a resilient tube, between the inlet end and outlet end of which is disposed the valve means, and the resilient tube and the valve means may be integral. The valve means may conveniently be made up of a deformable region in the wall of the tube and actuator means for pressing the deformable region thereby to wholly or partially close the tube thereby to prevent or inhibit, as the case may be, the flow of liquid from the outlet end of the tube.

Preferred embodiments of the invention therefore provide a device for controlling and managing the operation of a catheter and the measuring, recording, assessing, and interrogation of the physical and chemical parameters of the contents of the catheter, including the downloading of such information to a suitable memory, such as to a computer for storing, analysing etc such information and the subsequent controlled dispensation of appropriate drugs or medicines into the catheter.

The valve means may be actuated by any suitable means, such as hydraulic, pneumatic, mechanical or electrical means.

The means for assessing, measuring, recording, interrogating and downloading information relating to the contents of the urinary bladder during catheterisation may conveniently be electronic.

The means for dispensing appropriate drugs or medicines may be activated electronically or manually.

Where the catheter control unit is used to allow urine to pass from the bladder of a person, the apparatus is conveniently constructed in two parts:—
a) a one-use disposable tube with a region or regions which come into contact with urine, and
b) a reusable electronic control unit which operates the valve or valves, measures pressure, pH value of the urine and any other desired characteristic, and is capable of recording and assessing, downloading and interrogating such data etc.

Where two or more valves are used they may define chambers between them and means for sterilising/cleansing the valve chamber/chambers may be provided. The catheter control unit preferably includes means for measuring, recording and altering the pH value of the urine.

The control unit may conveniently be powered by a renewable or rechargeable battery.

The electronic means for storing, measuring, assessing, recording and early warning may be one or more microchips.

The disposable tube and/or the reusable part of the apparatus may be provided with a means for attaching it to a catheter and a drainage bag. Alternatively, urine may be discharged directly from the disposable tube rather than through a separate drain tube or into a drainage bag. The disposable tube and the reusable part of the apparatus may be provided with an automatic means of disconnection from the drainage bag if the drainage bag/tube is accidentally pulled. This may be by means of a link which is arranged to be broken when subjected to a predetermined longitudinal force.

In use the disposable tube and the or each deformable wall regions making up a valve may fit into the reusable electronic unit and be controlled by an electronic chip. When assembled together into one unit (collectively “urinary control apparatus”) the valve unit and reusable electronic unit can then automatically control the flow of urine from the bladder and monitor and record desirable information for interrogation. A manual override may be applied to control the flow of urine from the bladder if desired.

The urinary control apparatus may be designed to fit onto the open end of a urinary catheter such as a Foley catheter after the Foley catheter has been installed in the usual way in the bladder. A standard drainage bag may then be fitted to the outlet end of the tube if desired and draining of the bladder can take place in the usual way.

The urinary control apparatus, when supplied with a first electronically controlled valve, which desirably will be placed above the second electronically controlled valve on the catheter side of the control unit, can be used to regulate the flow of urine from the bladder when the sensing means, which may be a pressure switch, detects there is a predetermined pressure of urine in the bladder.

In a particular embodiment, at a predetermined pressure or time interval, the first electronically controlled valve will open and let out the urine from the bladder, after which it will close and urine will once again collect in the catheter tube and the bladder until the pressure/time builds up or time elapses ready for the next evacuation. Urine discharged through the urinary control unit may be collected in a standard collecting bag which can be fitted to the distal end of the urinary control apparatus.

When urine passes through a first open valve it may be monitored by the electronic unit, e.g. the pH value of the urine may be detected as well as the frequency and volume etc. The information may be monitored and an audible warning activated if an alarm condition is detected, so that remedial action may be taken.

The urinary control apparatus may be supplied with a second valve which is placed below the first valve on the drainage bag side. This provides a barrier chamber between the two valves in which e.g. a slow release anti-bacterial compound or the like may be sited. At a predetermined pressure/time interval the first and second valves open and let out urine from the bladder, after which they close and urine will once again collect in the catheter tube and the bladder until the pressure builds up/time elapses ready for the next evacuation. When the second valve is closed the slow release antibacterial compound will then collect in the barrier chamber to provide a barrier between the drainage bag and the urinary control apparatus. This helps to prevent bacteria travelling up into the first chamber and the catheter lumen. The provision of a chamber with an antibacterial compound or the like can therefore help to prevent infection of the bladder.

An electronic control unit can be used to control the operation of the valves and measure the physical and chemical characteristics of the urine as it passes through the unit. This will supply the data which is required for the early identification of adverse conditions affecting the patient and the catheter e.g. if the pH of the urine is monitored and recorded on a regular long term basis it is possible to identify those patients that are at risk of catheter encrustation and the subsequent risks of catheter blockage, trauma and infection to the wearer of the catheter.

It is preferred that the electronic information relating to the contents of the urinary bladder be recorded to a SMART card SIM card or similar capable of being removed from the apparatus and inserted into an appropriate card reader connected to a lap top, palm top PC or similar for downloading the information and reprogramming the card for insertion back into the apparatus.

Alternatively an infrared input output device may be used for communicating data relating to the contents of the urinary bladder during catheterisation to a suitable receiving device.

For measuring pH values various means are known in industry, such as by the use of electrodes. Alternatively, litmus paper or other pH reactive materials may be used. In one embodiment a solid state ISFET sensor is used.

The urinary control apparatus aspect of the current invention may also be beneficially provided with a flow meter which may preferably be of a Venturi type including one or more pressure sensors positioned within the resilient tube forming part of the apparatus. Preferably, the pressure sensors are electronic.

The urinary control apparatus may also be beneficially provided with means for measuring the colour and turbidity of the urine, this being achieved by using a light source projected e.g. through a thin-walled substantially transparent section of the disposable tube part of the apparatus onto a light sensor, such as an electronic light sensor.

The urinary control apparatus may also conveniently be provided with means for back flushing of the catheter which may be achieved via a sealable port on the disposable tube section of the apparatus. Alternatively, back flushing may be provided by the provision of means for generating a short term reverse flow of liquid, including urine in the form of a pulse within the catheter. The pulse may be rapidly repeated to loosen and dislodge encrustation at the tip of the catheter and so prevent build-up and subsequent blocking of the catheter. Preferably, the means for providing back flushing pulses is by rapidly repeated compression and relaxation of the walls of the resilient tube.

The urinary control apparatus may also be beneficially provided with a sterilising light source and means for dispensing sterilising light within the catheter and drainage bag system. Preferably, the sterilising light source produces ultraviolet light which may conveniently be dispensed within the catheter and drainage bag system by means of a ‘leaky’ type of optical fibre, in which case it is further preferred that the optical fibre be loosely inserted in the lumen of the catheter.

The benefits of placing the urinary catheter control apparatus between the catheter and the storage bag in certain preferred embodiments are as follows:—
a) the control apparatus can fit between an e.g. standard Foley catheter or standard supra-pubic catheter and standard drainage bag such that no, or minimal, user education is necessary;
b) the bladder can be controlled i.e. filled and emptied without any patient intervention;
c) the apparatus is suitable for use for a patient who is not conscious or for the elderly confused;
d) drainage of the bladder can be monitored and data relating to urine characteristics measured and recorded;
e) bladder drainage can be made automatic such that normal function of the bladder is maintained;
f) the problems associated with infection and crystallisation experienced with continuous drainage can be substantially reduced;
g) the provision of a barrier chamber and the regular flushing of the catheter can mean the possibility of less infection transmitting through the catheter lumen; and
h) there can be two seals between the storage bag and the catheter i.e. one at the catheter output end (the input of the urinary control apparatus) and the one way seal adjacent to the collection, which is usually installed to prevent backflow from it to further reduce the incidence of infection.

Substantially complete control of bladder drainage may therefore be possible through the use of the urinary control apparatus in preferred embodiments of the invention Bladder drainage can be continuous or intermittent, according to requirements. The control apparatus can be used for many different types of catheters and drainage bags since the attachments are usually universal. The urinary control apparatus can be made fail-safe in that it can give warning of failure. In such an event the apparatus can be removed simply by pulling it out of the catheter opening and the bag opening in the same way as the drainage bag fitting is removed from the catheter. The catheter may then be connected to the drainage bag in the usual way and bladder drainage resumed without the urinary control apparatus in place.

In an alternative embodiment of the invention the storage bag may not be attached to the urinary control apparatus but instead the valve or valves are operated manually or electronically in response to pressure sensing/time interval sensing if desired. This enables the patient to control and monitor the discharge of urine without the need for carrying a drainage bag.

The urinary catheter control apparatus can therefore be versatile in that a patient in hospital may be fitted with a standard catheter, a urinary control apparatus and a drainage bag. The urinary control apparatus may then be operated in conjunction with a drainage bag whilst the patient requires such treatment. However, when the patient is in a position to be discharged from hospital, or does not require a drainage bag any longer, after some simple user education, the drainage bag may be removed and the catheter and urinary control apparatus left in place. The patient will then be able to operate the discharge of urine manually through the urinary control apparatus and interrogate the electronic monitor to ascertain the pH level etc. of the urine. If the pH level of the urine needs to be altered an appropriate solution may be discharged into the catheter through the electronic control apparatus.

In another aspect there is provided a catheter control apparatus including a flow passage for being positioned between a catheter and a drain, and means for exposing to flow therethrough an active agent such as e.g. an acidifier or anti-microbial.

In a further aspect there is provided a catheter control apparatus including a flow passage for being positioned between a catheter and a drain, and means for imparting a pulsed reverse flow in said flow passage.

In another aspect there is provided a catheter control apparatus including a flow passage for being positioned between a catheter and a drain, and an optical waveguide for introducing sterilising radiation e.g. U.V. radiation into said flow passage.

In another aspect there is provided a catheter control apparatus including a flow passage for being positioned between a catheter and a drain, and a release mechanism for yielding or rupturing if a tension above a preset threshold is applied thereto.

In another aspect there is provided a catheter control apparatus including a flow passage for being positioned between a catheter and a drain, including means for sensing one or more parameters of the flow in use along said passage, and means for storing and/or transmitting said parameters.

In yet another aspect there is provided a catheter control apparatus including a flow passage for being positioned between a catheter and a drain, means for sensing the pH of the fluid therein, and means for using the sensed measurement to determine an indication of the service life of the catheter.

In a further aspect there is provided a catheter control apparatus including a flow passage for being positioned between a catheter and a drain, means for sensing the pH of the fluid therein, and means for releasing an acidifying agent in response to the sensed pH passing or tending towards a preset threshold.

In a further aspect, there is provided a method of controlling the flow of fluid along a catheter, which comprises sensing at least one parameter of the fluid in a test section of the flow passage and adjusting the flow therealong in response to said sensed parameter or parameters.

Whilst the invention is described above, it extends to any inventive combination of features set out herein.

The invention will now be described, by way of example only, with reference to the accompanying drawings, in which:

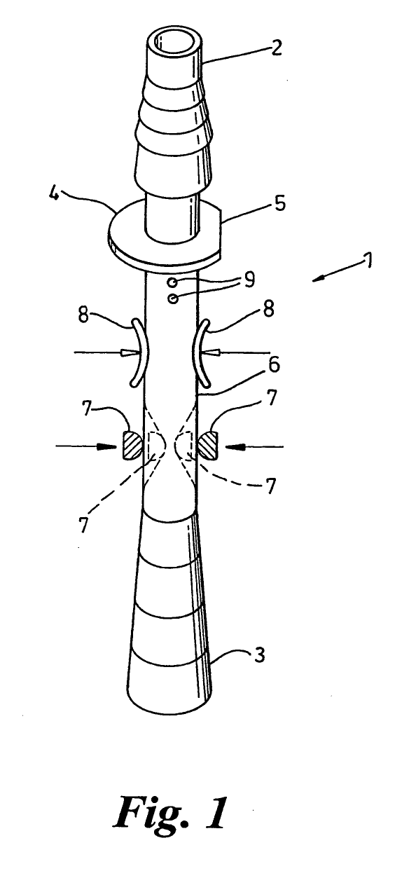

FIG. 1 is a general arrangement diagram of an inlet and outlet tube, associated valve means and sensing means of urinary catheter control apparatus;

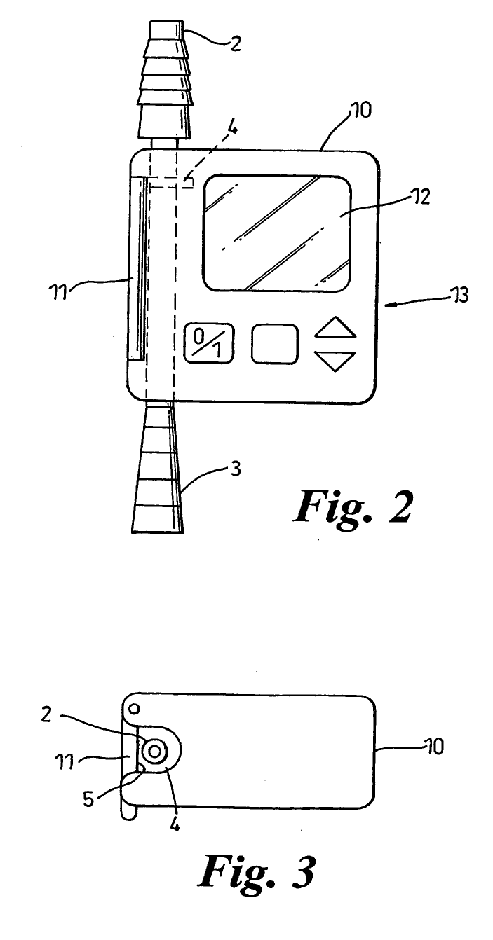

FIG. 2 is a general arrangement diagram of the assembled urinary catheter control apparatus of the invention;

FIG. 3 is a general arrangement drawing showing the plan view of the apparatus of FIG. 2;

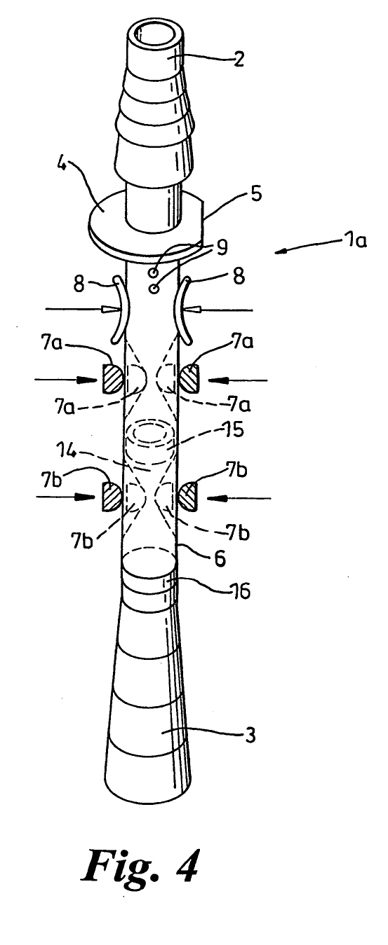

FIG. 4 is a general arrangement diagram of the tube of FIG. 1 modified to provide a double valving and sterilisation chamber together with a link arranged to be broken when subjected to a predetermined longitudinal force;

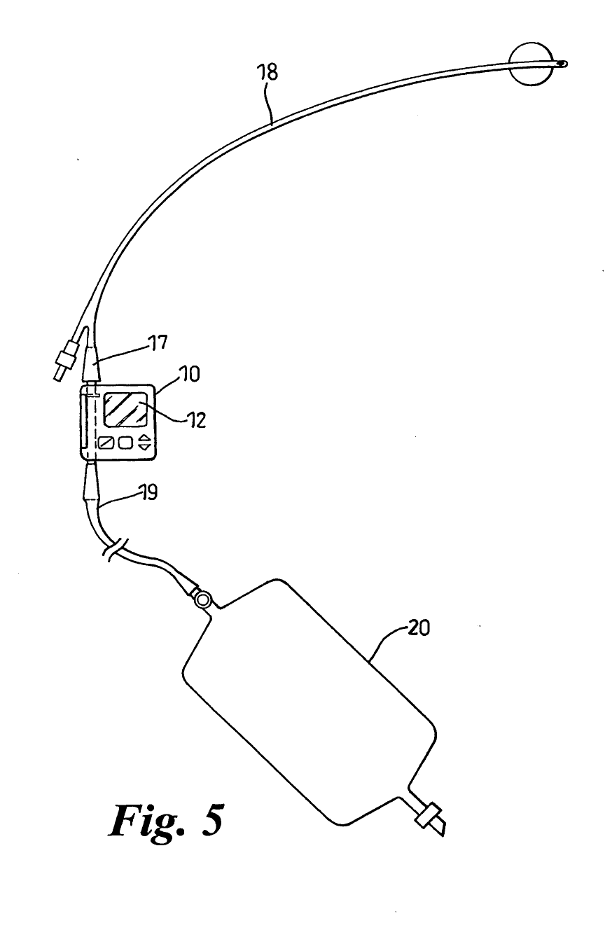

FIG. 5 is a general arrangement diagram of urinary catheter control apparatus of the invention attached to a typical urinary catheter and a typical urine collection bag; and

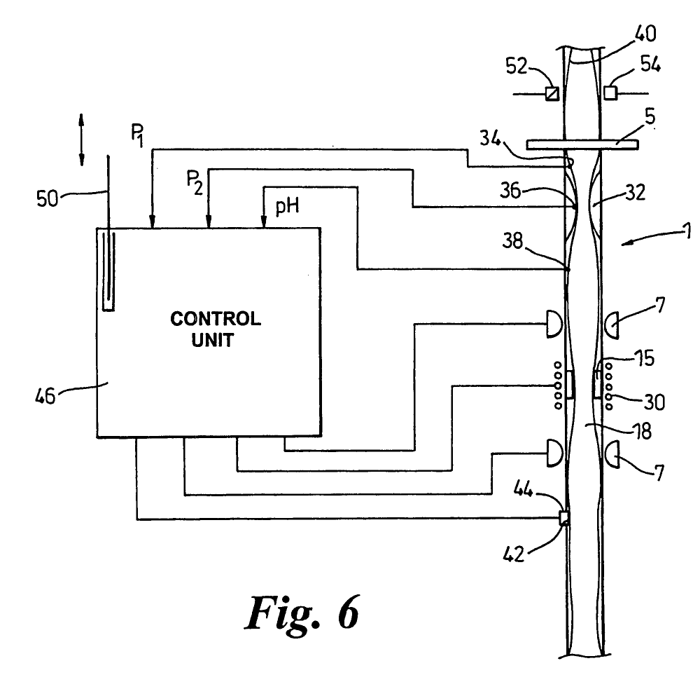

FIG. 6 is a schematic view of a further embodiment of this invention.

Referring firstly to FIG. 1, this shows part of a urine catheter control apparatus according to the invention which includes a resilient tube shown generally at 1, which may be made of e.g. silicone. The tube 1 includes at one end a frusto-conical male push connector 2 sized to fit into the cone connection end of a typical urinary catheter (not shown). At the other end of the tube 1 is a frusto-conical female connector 3 sized to allow the insertion and retention of a tube from a urine collection bag (not shown). Adjacent to the male connector 2 is a generally disk-shaped location flange 4 provided with a flattened region 5 on one side thereof so as to locate the tube 1 correctly within the urine collection apparatus in a manner to be described.

In the mid-region 6 of the tube 1 the walls thereof are deformable to the extent that two pairs of spaced of actuators 7a, 7b, when moved towards each other in the direction shown arrowed to the position shown in broken line can squeeze the tube 1 in this region sufficient to effect a valve arrangement by which flow of liquid therethrough may be prevented or inhibited.

In order to sense the pressure in the tube 1 a pair of pressure sensors 8 can be disposed in the manner as shown to lightly bear against the tube 1 in this region so that the internal pressure of liquid in the tube can then be determined to detect build-up of urine in the bladder of the catheter wearer when the actuators 7 are closed to their position shown in dotted outline, to prevent flow through the tube 1.

Above the pressure sensors 8 are a pair of electrical contacts 9 emanating from an ion sensitive field effect transistor pH sensor of known form built into the wall of the tube 1. This can therefore be used to sense the pH of liquid in the tube via electronic means in a manner to be described. Alternatively a conventional pH sensor may be used.

Referring now to FIGS. 2 and 3, there is shown the general arrangement of a catheter control apparatus in the form of urine catheter control apparatus comprising, in this embodiment, a housing 10 for containing electronic components including control components for the apparatus, as well as the tube 1, this being retained in place in the housing 10 by the flanged disk 4 being received within a correspondingly shaped recess in the housing 10 in a manner more clearly shown with reference to FIG. 3, in which it will be seen that a hinged door 11 can be used to trap the disk 4 and hence the tube 1 in position by bearing up against the flattened portion 5. The housing 10 supports the actuators 7a, 7b the pressure sensor 8 and the electrical circuitry for connection and to the pH sensor integrated in the tube 1.

A graphic display 12 is provided on a front face of the housing 10, which may be an LCD display, to give a visual data output of the apparatus, and control buttons shown generally at 13 are provided for actuating electronic components within the housing 10, such as for sensing, actuating and recording purposes.

FIG. 4 illustrates a second embodiment in which a tube shown generally at 1a is used which is similar to the tube 1 of FIG. 1. But in this instance, instead of having room for a single valve means including a single pair of actuators 7 as shown in FIG. 1, there are instead two pairs of actuators 7a and 7b displaced axially along the length of the middle portion 6 of the tube 1a, and corresponding other parts of the arrangement to that shown in FIG. 1 having corresponding numbering. As can be seen, when the pairs of actuators 7a and 7b are moved inwardly in the direction shown arrowed to the position shown in dotted outline a closed chamber 14 is formed between them into which may be inserted during the manufacture of the tube 1a a pellet 15 (also shown in dotted outline), such as in the shape of an o-ring, of a slow dissolving material capable of providing a sterilising action to any liquid in the chamber, the pellet being formed by commonly available bleaching or sterilising agents. Similarly, one or more other pellets may be releasably held in the tube 1 above the actuator 7 and may be capable of e.g. releasing acidity into the tube 1 and hence into the catheter lumen to help counter the build-up of alkalinity within the catheter resulting from urease bacteriological activity. The rate of dissolving may be controlled externally as required by providing, for example, a localised heating element in this region of the tube 1 with the pellet 15 being designed to release more or less acidity by varying the heating of the tube 1 and hence the pellet 15 in this region.

As a safety measure a breakable link 16 is provided above the female connector 3 and below the mid-section 6 of the tube 1, the link 16 having a weakened portion designed to snap when subjected to a pre-determined longitudinal force caused by accidental pulling or tension on the drainage bag and associated tubing.

In FIG. 5 there is shown a general arrangement of the urinary control apparatus of this embodiment of the invention attached by its male connector to the female connector 17 of a typical Foley-type urinary catheter 18, and is also attached by its female connector to the tube 19 (only part of which is shown) of a typical urine collection bag 20.

In use a control processor within the housing monitors the pH in the tube and controls cycling of the device to allow urine to pass from the catheter to a drainage bag, in accordance with preset criteria such as the sensed pressure exceeding a preset amount or elapse of a set time. The control processor may log details of the pH readings to a log. The log may be stored on a removable smart card or the like to allow the data to be downloaded to a remote medical attendant.

Furthermore the processor may apply the pH readings to a suitable algorithm to predict when the catheter needs changing, i.e. in good time before excessive encrustation or blockage. The algorithm may effectively integrate the area under a graph of pH vs time and flag a renewal warning when the area exceeds a preset amount. The area threshold may be determined empirically for various users. The unit may also flag a warning if the pH moves outside acceptable bounds, for example if it is too high.

Catheter control instructions may be downloaded from a remote centre onto the smart card which is then applied to the unit to control the catheter in accordance with these instructions.

Referring to FIG. 6 there is shown a schematic view of an embodiment of a catheter control device in accordance with this invention.

In this invention a resilient tube 1 is used similar to that of the previous embodiments, in conjunction with two spaced actuators 7 which are selectively operable to control the flow through the tube and also to create between them a closed chamber 14 in which is located a slow release ring 15 of an acidifying agent. The rate of release of the agent may be raised by heating it by means of a heating element 30 which passes around the outside of the tube.

The tube also includes a Venturi flow meter 32 upstream with pressure sensors 34, 36 at the throat and upstream thereof in customary fashion. The pressure sensors 34 and 36 may be any form of suitable pressure transducer which provides an output signal.

An ISFET pH probe 38 is disposed upstream of the upstream actuator 7. A lossy optical fibre 40 passes along the inside of the tube 1 and attaches to the wall of the tube at an external optical coupling region 42 by which sterilising U.V. light may be introduced into the fibre 40 from a U.V. source 44.

A microprocessor based control unit 46 receives, processes and stores data from the various sensors to determine parameters including pH, flow rate (from the venturi flow meter) and pressure, and uses these to apply a control function to the actuators 7, the heating element 30 and the U.V. steriliser source 44.

The microprocessor is pre-programmed to operate the apparatus according to a selected regime, and may be reprogrammed in use as to be described below.

Pressure-Related Operation

In a pressure-related operation, the control unit 46 may open both the valve actuators 7 when the static pressure in the catheter exceeds a preset threshold. Likewise the control unit may close the valve actuators once the sensed pressure and/or flow rate falls below a preset threshold.

Timed Operation

In addition the control unit 46 may open the valve actuators 7 at predetermined time intervals.

Cleaning/Flushing Operation

At predetermined intervals or in response to the pH falling below a preset threshold the control unit will initiate a cleaning flushing cycle. In this cycle, the valve actuators are operated to cause the chamber between them to fill with urine and the chamber is then closed. The heating element 30 is optionally operated to increase the rate of release of the active agent into the chamber 14. Once a sufficient amount of acidifying agent has been released, the downstream actuator 7 is kept closed whilst the upstream actuator is repeatedly applied and released to exert a pulsing action which causes the acidified charge of urine from the chamber to permeate the length of the catheter and in particular to the eye in the tip of the catheter where the risk of encrustation is normally greatest. The pulsing action not only distributes the dose of acidified urine to permeate along the catheter, but it can also exert a mechanical dislodging action to the encrustation or biofllm on the catheter. The cycle then concludes. During the period the U.V. source may be activated to expose the column of liquid in the tube 1 to U.V. radiation.

Data Logging and Programming

The microprocessor also keeps a log of the readings of pH, pressure etc and may apply the pH readings to an algorithm which effectively calculates the area under the pH vs time graph and compares this to a threshold value which is indicative of the catheter nearing the end of its service life. When this threshold is exceeded, the control unit flags an alarm to the user so that they can arrange for the catheter to be changed in good time before the performance thereof deteriorates.

The log of the readings is stored on a replaceable smart card or “sim” card 50 which can be removed from the unit and read by a standard card reader. This data can be transmitted from the card reader to a remote medical practitioner, e.g. by a modem and telephone line, so that they can monitor the parameters. In addition, programming instructions can be sent to the control unit from the central station along the same link so that the operation of the device can be changed, for example if the parameters received by the medical practitioner indicate that a different cycling regime is called for. Instead of using a smart card, other suitable communications technology may be used.

In this way a medical practitioner such as a district nurse may monitor many different users and adjust their regime remotely with a considerable saving in time.

Also shown in the arrangement of FIG. 6 are an optical transmitter 52 and an optical receiver 54, which may be connected to and controlled by said control unit 46 to detect the colour and or turbidity of the urine in the tube. These readings may be used alone or jointly with other readings to modulate operation of the flow control actuators 7.

In a further embodiment of the invention there is provided a means for controlling and managing an intravascular device. In this embodiment, the electronic control circuitry and associated tube may be used to measure internal pressure at the site of an intravascular installation and if pressure rises beyond a set limit, e.g. the onset of “tissuing”, one or more valves may operate automatically to limit the flow and thus reduce the pressure in the intravascular device. At the same time, a warning sound or light may be activated. In this embodiment it is preferred that the pressure sensor be positioned after the valve or valves in order to sense the pressure in the intravascular device, which may typically be a venflon.

1. Apparatus for controlling the flow of fluid to or from a human or animal body, the apparatus including:—
a flow passage having an inlet and an outlet,
respective spaced flow control means disposed between the inlet and the outlet for controlling the flow of liquid through the apparatus and defining between them at least one chamber region,
at least one sensing means for sensing one or more properties of the liquid in or adjacent the apparatus in use, and
control means for controlling operation of the flow control means in response to at least one of:
(i) a sensed property of the liquid, and
(ii) elapse of a predetermined time interval.

2. Apparatus according to claim 1 wherein said flow passage is defined by a tube of resilient material, between the inlet end and outlet end of which are disposed the first and second flow control means.

3. Apparatus according to claim 1 wherein the tube of resilient material includes a generally smooth and continuous inner surface.

4. Apparatus according to claim 2 wherein each of the flow control means comprises an actuator for resiliently deforming the local wall region of the tube to press opposite regions towards each other thereby to wholly or partially close the tube thereby to prevent or reduce as the case may be, the flow of liquid through said tube.

5. Apparatus according to claim 4 wherein said flow passage is located in the flow path between a catheter and a drainage tube or bag.

6. Apparatus according to claim 5, wherein said tube of resilient material is a disposable item which is removably located in a housing, which supports said actuators.

7. Apparatus according to claim 6, wherein at least one of said sensing means is also located in said housing.

8. Apparatus according to claim 1, wherein said sensing means includes a pressure sensor.

9. Apparatus according to claim 1 when said sensing means includes a pH sensor.

10. Apparatus according to claim 9, wherein said pH sensor comprises a pH probe exposed to the fluid in use in said flow passage.

11. Apparatus according to claim 10, wherein said pH probe is an ion sensitive field effect transistor (ISFET) probe.

12. Apparatus according to claim 1 wherein said sensor means include a flow rate sensor.

13. Apparatus according to claim 12, wherein said flow rate sensor comprises a venturi within said flow passage.

14. Apparatus according to claim 1, wherein said sensor means includes an optical sensor for detecting at least one of the colour and turbidity of the liquid in use in said flow passage.

15. Apparatus according to claim 1 including means for storing data sensed by at least one of said sensing means.

16. Apparatus according to claim 1 wherein said control means are programmable.

17. Apparatus according to claim 1, including data transfer means for transferring data or instructions between at least one of
(iii) said sensor means and
(iv) said control means and
(v) an external device.

18. Apparatus according to claim 17, wherein said data transfer means is a removable data storage device.

19. Apparatus according to claim 18, wherein said removable data storage device is a smart card, sim card or the like.

20. Apparatus according to claim 17, wherein said data transfer means is an interface device for communicating across a public communications network.

21. Apparatus according to claim 17, wherein said data transfer means is an infra red input output device.

22. Apparatus according to claim 9, including means for exposing fluid in said chamber region to an active agent.

23. Apparatus according to claim 22, wherein said active agent is a pH modifier.

24. Apparatus according to claim 22, wherein said active agent is an anti-microbial agent.

25. Apparatus according to claim 22, wherein said active agent is a cleansing agent.

26. Apparatus according to claim 22, wherein said means for exposing comprises a pellet or block of said active agent.

27. Apparatus according to claim 26, including means for accelerating dispersion of said active agent.

28. Apparatus according to claim 27, wherein said acceleration means comprise an electric heater element.

29. Apparatus according to claim 23, wherein said control means is responsive in use to the output of the pH sensor to control in use the local pH within the flow path.

30. Apparatus according to claim 6, characterised in that the disposable tube and/or the reusable part of the apparatus is provided with means for attaching it to the catheter and a drainage bag.

31. Apparatus according to claim 6, further characterised in that the disposable tube and the reusable part of the apparatus is provided with an automatic means of disconnection from the drainage bag if the drainage bag/tube is accidentally pulled.

32. Apparatus according to claim 31 further characterised in that the automatic means of disconnection is by means of a link adapted to be broken when subjected to a predetermined longitudinal force.

33. Apparatus according to claim 6 characterised in that the disposable tube and valve means are adapted to fit into the reusable electronic unit.

34. Apparatus according to claim 33 further characterised in being provided with a manual override to control the flow of urine from the bladder.

35. Apparatus according to claim 5 further characterised in that at least one of said first and second flow control means is actuated by said control means to regulate the flow of urine from the bladder when the sensing means detects there is a predetermined pressure of urine in the bladder.

36. Apparatus according to claim 2 further characterised in that the sensing means comprises a pressure switch.

37. Apparatus according to claim 5, further including means for back flushing of the catheter.

38. Apparatus according to claim 37 further characterised in that the means for back flushing of the catheter is via a sealable port on the disposable tube section of the apparatus.

39. Apparatus according to claim 37 further characterised in that the means for back flushing of the catheter is by the provision of means for generating a short term reverse flow of liquid in the form of a pulse within the catheter.

40. Apparatus according to claim 39, further characterised in that the means for providing back flushing pulses is by rapid repeated actuation and release of one of said actuators.

41. Apparatus according to any one of the preceding claims claim 2 further characterised in that a sterilising light source is provided.

42. Apparatus according to claim 41 further characterised in that the sterilising light source produces ultraviolet light.

43. Apparatus according to claim 41, further characterised in that the sterilising light source is dispensed within the catheter by means of a “leaky” type of optical fibre.

44. Apparatus according to claim 43 further characterised in that the optical fibre is loosely inserted in the lumen of the catheter.

45. A disposable tube for use in the apparatus of claim 6, said tube having an inlet end and an outlet end and resiliently deformable sidewalls by which flow of liquid therethrough may be controlled via sidewall pressure thereon, characterised in that the tube is adapted to be received within said housing.

46. A tube according to claim 45 further characterised in that it includes location means enabling it to be correctly oriented within the housing.

47. A tube according to claim 46 further characterised in that the location means comprises a flange having a flattened region adapted to rest against a correspondingly flattened region of the housing.

48. A tube according to claim 45 further characterised in that it includes an integral pH transducer and associated electrical contacts by which the pH of liquid passing therethrough may be monitored.

49. A tube according to claim 45 further characterised in that it includes a weakened region in the form of a breakable link adapted to snap when subjected to a predetermined longitudinal force, relative to the major axis of the tube.

50. A slow release pellet adapted to be inserted within a tube according to claim 45, the pellet comprising or including a sterilising and/or cleansing agent.

51. A pellet according to claim 50, further characterised in that it is in the form of a hollow ring dimensioned to fit within the tube and to allow liquid to flow therethrough.

52. A method of controlling the flow of fluid along a catheter, which comprises sensing at least one parameter of fluid in a test section of the flow passage and adjusting the flow therealong in response to said sensed parameter or parameters.

53. A method according to claim 52, which includes detecting the pH at intervals and applying the measurements of pH to an algorithm which provides an indication of the service life of the catheter.

54-55. (canceled)
